(19) United States
(12) Patent Application Publication
(10) Pub. No.: US 2010/0178278 A1
(43) Pub. Date: Jul. 15, 2010

(54) FORMABLE BIOCERAMICS

(75) Inventors: Tzy-Jiun Mark Luo, Cary, NC (US); Ching-Chang Ko, Chapel Hill, NC (US); Camilla Tulloch, Chapel Hill, NC (US)

Correspondence Address: MOORE & VAN ALLEN PLLC, P.O. BOX 13706, Research Triangle Park, NC, 27709, US

(73) Assignee: THE UNIVERSITY OF NORTH CAROLINA AT CHAPEL HILL, Chapel Hill, NC (US)

(21) Appl. No.: 12/685,743

(22) Filed: Jan. 12, 2010

Related U.S. Application Data
(63) Continuation-in-part of application No. PCT/US08/69923, filed on Jul. 14, 2008.
(60) Provisional application No. 60/949,281, filed on Jul. 12, 2007.

(51) Int. Cl. A61K 35/12; A61K 33/42; A61P 43/00

(52) U.S. Cl. 424/93.7; 424/602; 977/831

(57) ABSTRACT

A formable bioceramic including hydroxyapatite nanocrystals, gelatin, and sol-gel-containing material is described. Also described is a process for making and using the bioceramic. The formable bioceramic displays superior mechanical strength, elasticity, biocompatibility and forming capabilities and is targeted for bone repairs and template-assisted tissue engineering applications.

### FIELD

This invention relates generally to a formable bioceramic, and more particularly to a sol-gel based hydroxyapatite-gelatin bioceramic (GEMOSOL), and even more particularly to a aminosilica-based hydroxyapatite-gelatin bioceramic (GEMOSIL).

### DESCRIPTION OF THE RELATED ART

Many different materials have been used for bone replacement and substitution, however, to date the materials used have not performed as well as natural bone. These bone substitutes have not been ideal because they have very different mechanical properties and often exhibit less than desirable biocompatibility.

Attempts at bone replacement have used a variety of foreign materials, with resulting associated problems. Metals that have been used to replace bone structure, such as stainless steel and titanium, have been found to mechanically mismatch with properties of bone to which they are implanted or attached. Additionally, these materials often cause allergic reactions and inflammation due to abrasive particles and leached ions such as Nickel, Cobalt, Chromium, Aluminum, and Vanadium ions. Teflon joint implants have been used, but have been known to shatter and erode when used in applications requiring repetition and force, such as use as jaw implants. Bio-inert materials such as alumina and zirconia ceramics exhibit many of the same clinical problems associated with metal implants.

Other approaches have used many of the same materials as found in natural bones in an attempt to create more viable and long lasting bone replacement materials. Natural bones are an extracellular matrix mainly composed of hydroxyapatite crystals and collagen, with the hydroxyapatite well-mineralized on collagen at body temperature. The strength of the hydroxyapatite/collagen bonding and the quality and maturity of the collagen fibers are important for the mechanical properties of bone. Therefore, many of these attempts have focused on developing hydroxyapatite and collagen mixtures for bone substitutes, however, collagen is an expensive material, and the reaction of collagen with hydroxyapatite can be difficult to control. This lack of control has led to materials having reduced and/or inconsistent physical strength.

Implants using cement and ceramic materials, such as calcium phosphate, have also been made. These cements and ceramics overcome many of the problems noted above, as they can directly connect with bone and do not exhibit the reactions and inflammation common to many other implants. Additionally, as these materials are biocompatible, natural bone material grows slowly into the implants over time. However, these cements and ceramics are brittle, often have poor flexture strength, and are weak in energy absorption. Also, the materials used have generally been difficult to sculpt, leading to problems with irregular defects, and granule migration from the implant site. Therefore, these materials have not been widely used, and when used, have generally been limited to non-load bearing indications.

Natural bone, either large pieces or compositions, have also been used, with compositions using aggregates of bone particles receiving a high level of interest. The objective has been to more closely mimic natural bone and increase the strength of the implant. This also retains biocompatibility and allows bone ingrowth and assimilation. However, there are problems with harvesting and availability of bone components. Additionally, there are risks and complications associated with bone grafts or compositions, including risks of infection, viral transmission, disease, rejection, and other immune system reactions.

In addition to bone replacement, attempts have also been made to replace other bodily tissues. Various attempts have used animal tissues to replace human tissues, have used tissues from other locations in the body, or have attempted to use synthetic materials. These methods all have associated drawbacks and shortcomings.

Accordingly, there exists a need for a synthetic implant material that is lightweight, strong, cost-effective, elastic, and which offers a high degree of biocompatibility, while exhibiting rapid integration with the surrounding tissues and structures. The material may be useful for applications including, but not limited to, repairs, replacement, template-assisted tissue engineering, and other engineering applications.

### SUMMARY

The present invention relates generally to novel composite bioceramics. More specifically, the present invention relates to sol-gel based hydroxyapatite-gelatin formable bioceramics and methods of making and using same.

In one aspect, a formable bioceramic comprising calcium phosphate/gelatin-modified silica (GEMOSIL) nanocomposite is described.

In another aspect, a formable bioceramic comprising calcium phosphate/gelatin-modified sol-gel (GEMOSOL) nanocomposite is described.

In another aspect, an article for use in tissue engineering is described, wherein the article comprises a formable bioceramic comprising calcium phosphate/gelatin-modified silica (GEMOSIL) nanocomposite and/or a calcium phosphate/gelatin modified sol-gel (GEMOSOL) nanocomposite.

In yet another aspect, an article for use in replacement is described, wherein the article comprises a formable bioceramic comprising calcium phosphate/gelatin-modified silica (GEMOSIL) nanocomposite and/or calcium phosphate/gelatin-modified sol-gel (GEMOSOL) nanocomposite. Preferably, replacement is selected from the group consisting of bone replacement, tooth replacement, joint replacement, cartilage replacement, tendon replacement, and ligament replacement.

In still another aspect, a method of making a formable bioceramic is described, said method comprising:
mixing calcium hydroxide, phosphoric acid and gelatin under aqueous conditions to produce a co-precipitated calcium phosphate-gelatin material; and
adding at least one silane reactant to the calcium phosphate-gelatin material to produce a calcium phosphate/gelatin-modified silica (GEMOSIL) nanocomposite.

In still another aspect, a method of making a formable bioceramic is described, said method comprising:
mixing calcium hydroxide, phosphoric acid and gelatin under aqueous conditions to produce a co-precipitated calcium phosphate-gelatin material; and
adding at least one sol-gel precursor to the calcium phosphate-gelatin material to produce a calcium phosphate/gelatin-modified sol-gel (GEMOSOL) nanocomposite.

In another aspect, a method of making a formable bioceramic is described, said method comprising:
mixing calcium hydroxide, phosphoric acid and gelatin under aqueous conditions to produce a co-precipitated calcium phosphate-gelatin material;
concentrating the calcium phosphate-gelatin material to remove excess water;
suspending the concentrated calcium phosphate-gelatin material in at least one alcohol;
concentrating the calcium phosphate-gelatin material to remove excess alcohol; and
adding at least one silane reactant to the calcium phosphate-gelatin material to produce a calcium phosphate/gelatin-modified silica (GEMOSIL) nanocomposite.

In another aspect, a method of making a formable bioceramic is described, said method comprising:
mixing calcium hydroxide, phosphoric acid and gelatin under aqueous conditions to produce a co-precipitated calcium phosphate-gelatin material;
concentrating the calcium phosphate-gelatin material to remove excess water;
suspending the concentrated calcium phosphate-gelatin material in at least one alcohol;
concentrating the calcium phosphate-gelatin material to remove excess alcohol; and
adding at least one sol-gel reactant to the calcium phosphate-gelatin material to produce a calcium phosphate/gelatin-modified sol-gel (GEMOSOL) nanocomposite.

In another aspect, a method of making a formable bioceramic is described, said method comprising mixing calcium phosphate-gelatin material with at least one silane reactant to produce a calcium phosphate/gelatin-modified silica (GEMOSIL) nanocomposite.

In another aspect, a method of making a formable bioceramic is described, said method comprising mixing calcium phosphate-gelatin material with at least one sol-gel reactant to produce a calcium phosphate/gelatin-modified sol-gel (GEMOSOL) nanocomposite.

Yet another aspect relates to a bioceramic, comprising implanting an article comprising a bioceramic, wherein the bioceramic comprises a calcium phosphate/gelatin-modified silica (GEMOSIL) nanocomposite and/or a calcium phosphate/gelatin-modified sol-gel (GEMOSOL) nanocomposite.

Still another aspect relates to a method of bone regeneration, comprising using a calcium phosphate/gelatin-modified silica (GEMOSIL) nanocomposite and/or a calcium phosphate/gelatin-modified sol-gel (GEMOSOL) nanocomposite.

Another aspect relates to a method of cartilage regeneration, comprising using a calcium phosphate/gelatin-modified silica (GEMOSIL) nanocomposite and/or a calcium phosphate/gelatin-modified sol-gel (GEMOSOL) nanocomposite.

Other aspects, features and embodiments will be more fully apparent from the ensuing disclosure and appended claims.

### BRIEF DESCRIPTION OF THE DRAWINGS

FIG. 1 is a representation of an embodiment of the formable bioceramic described herein.

FIG. 2 is a flowchart showing process steps for producing a bioceramic described herein.

FIG. 3 is a flowchart showing process steps for producing a bioceramic described herein.

### DETAILED DESCRIPTION, AND PREFERRED EMBODIMENTS THEREOF

A formable bioceramic is described that can be used as a replacement material for a variety of body applications. The formable bioceramic includes an intermixed and substantially uniformly dispersed composition including hydroxyapatite nanocrystals, gelatin fibers, and a sol-gel bioceramic network which intervenes with the hydroxyapatite-gelatin composites.

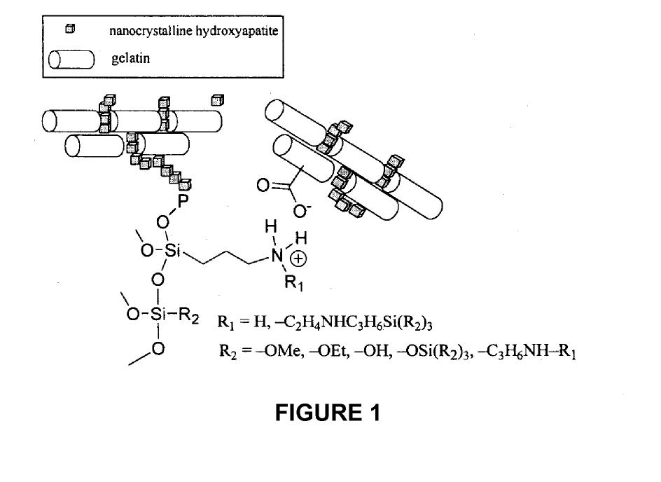

As shown in FIG. 1, which represents an embodiment described herein, hydroxyapatite nanocrystals are embedded into a matrix formed of silicon-containing chains and gelatin fibers. All of the components are substantially dispersed within the composite, resulting in relatively consistent properties throughout the composite. As defined herein, “substantially dispersed” and “substantially uniformly dispersed” corresponds to less than 10% variation in the chemical makeup throughout the composite, regardless of whether sampled interiorly or exteriorly, preferably less than 5% variation, and most preferably less than 2% variation.

Advantageously, the process described herein is based on the sol-gel process, wherein synthesis of the biomaterial from solution occurs at low temperatures, e.g., room temperature, which allows for the incorporation of biomolecules and living cells in said biomaterial. The sol-gel process is a wet chemical technique whereby a chemical solution undergoes hydrolysis and polycondensation reactions to produce colloidal particles (the “sol”) such as metal oxides. The sol will form an inorganic network containing a liquid phase (the “gel”). The “sol-gel” materials, as defined herein, include SiO₂, TiO₂, ZrO₂, and combinations thereof.

As defined herein, “silica” corresponds to SiO₂.

It has been discovered that gelatin can provide a bioactive surface to induce hydroxyapatite crystal growth. Suitable gelatins include both high bloom and low bloom gelatin. Preferably, gelatins having a bloom value between about 100 and about 300 will be used. “Bloom value” is a measurement of the strength of a gel formed by a 6 and ⅔% solution of the gelatin, that has been kept in a constant temperature bath at 10 degrees centigrade for 18 hours. The properties of the final bioceramic depend in part on the characteristics of the gelatin used. Variously, gelatin may be obtained that is produced from different animals, including cows and pigs. Gelatin may be extracted from various collagen-containing body parts, including bone and skin. The gelatin may be selected according to the desired application, as different gelatins, depending on the source and the extent of denaturation, may provide a better choice for the composite, depending upon the desired mechanical properties or biological activity level. Generally, it has been found that bovine gelatin provides better composites for many applications. An example of a suitable gelatin is standard unflavored gelatin (available from Natural Foods Inc., Canada). The gelatin may be dissolved into solution before use, preferably to form an aqueous solution. The gelatin may be used without purification or other prepatory steps.

In one aspect, a sol-gel-based hydroxyapatite-gelatin bioceramic including hydroxyapatite nanocrystals, gelatin and sol-gel-containing material is described. In another aspect, a silica-based hydroxyapatite-gelatin bioceramic including hydroxyapatite nanocrystals, gelatin and silica-containing material is described.

The gelatin may be modified prior to use in a reaction mixture. Preferably, the gelatin will be at least partially phosphorylated before use as a reactant. For example, the gelatin may be phosphorylated by the addition of phosphoric acid, ammonium phosphate ((NH₄)₃PO₄), diammonium hydrogen phosphate ((NH₄)₂HPO₄), ammonium dihydrogen phosphate (NH₄H₂PO₄), monoammonium phosphate (NH₄.H₂PO₄), or combinations thereof (available from chemical supply firms such as Fisher Scientific and Sigma Chemical) to a gelatin solution, or the gelatin may be added to a phosphoric acid solution. It is believed that phosphorylation leads to and enables better dispersion and growth of the hydroxyapatite nanocrystals. In solutions with phosphorylated gelatin, there will typically be excess phosphoric acid available for later crystal formation and/or growth.

The hydroxyapatite nanocrystals are formed through a reaction between phosphoric acid and/or phosphorylated locations on the gelatin fibers and calcium hydroxide. The phosphorylated locations are frequently the starting locations for hydroxyapatite crystal growth, however, hydroxyapatite crystal growth may also occur in solution between the phosphoric acid and calcium hydroxide components. These crystals may grow and embed themselves into the gelatin matrix structure by binding themselves to groups, such as carboxyl and amide groups, on the gelatin molecules. Once begun, the crystals grow by incorporating more calcium hydroxide and phosphoric acid components into the crystal. The product of this reaction includes a co-precipitated hydroxyapatite-gelatin colloidal material.

Calcium hydroxide is available from chemical supply firms such as Fisher Scientific and Sigma Chemical. However, calcium hydroxide may also be produced in a process including calcining calcium carbonate, which removes carbon dioxide to form calcium oxide. After calcining, the calcium oxide is hydrated to form calcium hydroxide. Following hydration, the calcium hydroxide may be weighed as a quality check. Due to the reactive nature of calcium hydroxide, and the tendency of calcium hydroxide to degrade quickly, special care should be taken with calcium hydroxide to ensure a high quality level of the calcium hydroxide. Because of this concern with the quality of the calcium hydroxide, producing calcium hydroxide just prior to use is preferred.

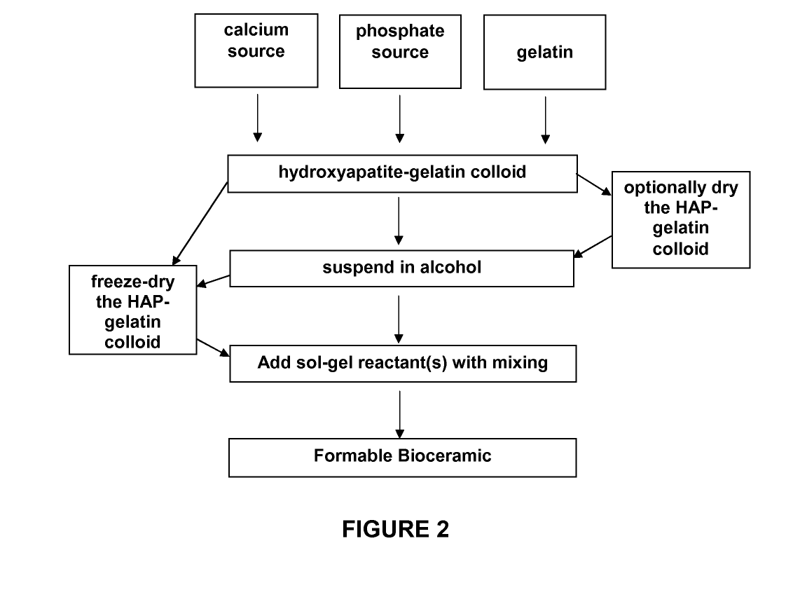

The hydroxyapatite-gelatin colloid may be incorporated into a sol-gel or silica matrix with or without removable active fillers and/or other additives to produce the formable bioceramic described herein, as shown schematically in FIG. 2. Although not wishing to be bound by theory, it is thought that the hydroxyapatite-gelatin colloid at least partially dissolves in the sol-gel or silica matrix, which creates a strong bond. Silane reactants contemplated for the sol-gel or silica matrix include, but are not limited to, tetramethylorthosilicate (TMOS), tetraethylorthosilicate (TEOS), 3-aminopropyltrimethoxysilane, bis[3-(trimethoxysilyl)propyl]-ethylenediamine (enTMOS), bis[3-(triethoxysilyl)propyl]-ethylenediamine, methyltrimethoxysilane (MTMS), polydimethylsilane (PDMS), propyltrimethoxysilane (PTMS), methyltriethoxysilane (MTES), ethyltriethoxysilane, dimethyldiethoxysilane, diethyldiethoxysilane, diethyldimethoxysilane, bis(3-trimethoxysilylpropy)-N-methylamine, 3-(2-Aminoethylamino)propyltriethoxysilane, N-propyltriethoxysilane, 3-(2-Amino ethylamino)propyltrimethoxysilane, methylcyclohexyldimethoxysilane, dimethyldimethoxysilane, dicyclopentyldimethoxysilane, 3-[2 (vinyl benzylamino)methylamino]propyltrimethoxysilane, 3-aminopropyltriethoxysilane, 3-(aminopropyl)dimethylethoxysilane, 3-(aminopropyl)methyldiethoxysilane, 3-(aminopropyl)methyldimethoxysilane, 3-(aminopropyl)dimethylmethoxysilane, N-butyl-3-aminopropyltriethoxysilane, N-butyl-3-aminopropyltrimethoxysilane, N-(β-amimoethyl)-γ-amino-propyltriethoxysilane, 4-amino-butyldimethyl ethoxysilane, N-(2-Aminoethyl)-3-aminopropylmethyldimethoxysilane, N-(2-Aminoethyl)-3-aminopropylmethyldiethoxysilane, 3-aminopropylmethyldiethoxysilane, or combinations thereof. Preferably, the silane reactant includes at least one amino-containing silane reactant. Titanium reactants contemplated for the sol-gel matrix include, but are not limited to, titanium isopropoxide. Zirconium reactants contemplated for the sol-gel matrix include, but are not limited to, zirconium ethoxide, zirconium propoxide, and zirconium oxide.

It is also contemplated herein that hydroxyapatite-collagen colloids, as well known in the art, may be incorporated into a sol-gel or silica matrix with or without removable active fillers and/or other additives to produce a formable bioceramic.

Importantly, the use of at least one sol-gel reactant results in the formation of a short-chain bioceramic oxide network with entrapped, substantially dispersed, hydroxyapatite-gelatin colloidal material. For example, at least one silane reactant results in the formation of a short-chain bioceramic silica network with entrapped, substantially dispersed, hydroxyapatite-gelatin colloidal material. Preferably, the at least one silane reactant includes at least one amino-containing silane compound. The aminosilane compounds provide enough binding strength to harness both the inorganic phase and the organic gelatin molecules. Moreover, when amino-containing silane compounds are used, the solidification reaction is more rapid. That said, for better control of the reaction speed and the final product, an amount of at least one non-amino containing silane compound may be included with the amino-containing silane compound(s). The rate of the solidification reaction and the control of the overall product may be controlled by adjusting the quantity of non-amino containing silane compound(s) relative to the amino-containing silane compound(s). Further, a silica-based network may further include titania and zirconia.

Inactive filler material includes, but is not limited to, poly(lactic-co-glycolic acid), poly(lactic acid), poly(glycolic acid), polyacrylic acid, poly(ethylene oxide), calcium phosphate, potassium chloride, calcium carbide, calcium chloride, sodium chloride, polystyrene, and combinations thereof. Some inactive fillers can be solidified with the GEMOSIL nanocomposite to serve as structural templates including, but not limited to, poly(N-isopropylacrylamide) and calcium chloride. Poly(N-isopropylacrylamide) may be removed from the bioceramic following formation of same by lowering the incubation temperature. Calcium chloride may be removed from the bioceramic following formation of same using water. These fillers may be removed as needed to create porous structures for biomedical applications.

With regards to porosity, salt leaching techniques, the introduction of bubbles (e.g., using an inert gas), and adding low temperature foaming agents are contemplated to control the pore size in the bioceramic.

Advantages associated with the novel sol-gel-based hydroxyapatite-gelatin bioceramic described herein include, but are not limited to, compatibility with carbon-based lifeforms, good mechanical strength similar to the hydroxyapatite-gelatin composite, better elasticity than conventional bioglass, excellent compressive strength, superb formability for scaffolding and upregulated cell differentiation.

In another aspect, a method of making a sol-gel-based hydroxyapatite-gelatin bioceramic using a sol-gel reaction that includes hydrolysis and condensation is described. In one embodiment, a method of making a silica-based hydroxyapatite-gelatin bioceramic using a sol-gel reaction that includes hydrolysis and condensation is described. The method of making said silica-based hydroxyapatite-gelatin bioceramic will be discussed hereinbelow.

Advantageously, the sol-gel method of making the biomaterial does not require a hydroxyapatite powder drying process which, if used, may result in excessive sample shrinkage, extended process times, and loss of materials. In addition, the process does not consume large quantities of hydroxyapatite-gelatin materials which results in a biomaterial having a substantially lower density than those previously reported. That said, a dry hydroxyapatite-gelatin colloid may be desirable depending on the desired product and the processing conditions.

Optionally, other components or additives may be added to the formable bioceramic. These additives may be added for various reasons. For example, additives may be added to increase biocompatibility, to decrease the possibility of rejection, to decrease the risk of infection, to increase the rate of natural bone growth in the bioceramic, or to increase the rate of natural cell growth near the implant. Additives may also be added to change or enhance some of the properties of the bioceramic. For example, the bioceramic may include growth factors, cells, other materials and elements, curing or hardening components, and other possible additives. Importantly, the sol-gel-based hydroxyapatite-gelatin bioceramic described herein can host additives on the surface or within the material.

Among other benefits, growth factors can assist in increasing natural growth, including the growth of natural tissues and bone into the area of the biomimetic nanocomposite. Examples of suitable growth factors include, but are not limited to, bone morphogenic protein (BMP), transforming growth factor (TGF-β) vascular endothelial growth factor (VEGF), matrix gla protein (MGP), bone siloprotein (BSP), osteopontin (OPN), osteocacin (OCN), insulin-like growth factor (IGF-I), Biglycan, Receptor activator of nuclear factor kappa B ligand (RANKL), and procollagen type I (Pro COL-α1), and combinations thereof.

Alternatively, cells may be added to the bioceramic in order to increase the rate of natural bone growth in the area of the bioceramic. Precursor cells may be added to the bioceramic to speed the rate of natural cell growth. Suitable cells include, but are not limited to, osteoblasts, osteoclasts, osteocytes, multipotent stem cells, and combinations thereof.

Optionally, other materials and elements may be added to the bioceramic. Elements and materials may be added to provide an additional feature, property, or appearance to the bioceramic, or for other reasons. Examples of suitable elements include fluoride, calcium, ions thereof, or other elements or ions. Examples of other suitable materials include polymers, ceramic particles, radio-opaque components, metals, and other materials. Variously, the bioceramic can include ceramic particles, fluoride, calcium, and/or a radio-opaque material.

As another alternative, curing additives may be added to the bioceramic. Suitable curing agents include chelating agents (e.g., water soluble polyalkenoic acids), photo- and uv-curable agents (e.g., UV-curable silane). A curing agent enables the bioceramic to harden more rapidly and allows the bioceramic to be used for a wider variety of uses. For example, a paste or viscous mixture of the bioceramic could be applied to an area of a bone or a tooth, and then rapidly cured to harden in place. This approach has the potential to improve the outcome and decrease patient recovery time.

Examples of other optional additives include growth inhibitors, pharmaceutical drugs, anti-inflammatory agents, antibiotics, and other chemicals, compositions, dyes, or drugs. These could be used in various applications of the bioceramic. For example, growth inhibitor may be used to prevent the ingrowth of certain undesirable cells, so that the bioceramic continues to function most effectively. Antibiotics may be used to decrease the likelihood of infection around the area of treatment. Pharmaceutical drugs, anti-inflammatories, and antibiotics may be used to reduce inflammation, minimize bleeding, increase healing, or for other uses.

The bioceramic may be used for a wide range of alloplastic uses, for a variety of purposes, and in a variety of applications. Alloplastic refers to synthetic biomaterials, in contrast to natural biomaterials which may be from the same individual (autogenic), from the same species (allogenic), or from a different species (xenogenic). The properties of the bioceramic may be modified to better meet the requirements of the use, purpose, or application for which it is intended. The properties depend in part on the gelatin used, the alignment of fibers and chains, the extent of nanoparticle formation and the stoichiometry of same, and the amount and type of silane reactant(s) used. Thus, the resulting bioceramic may have a wide range of mechanical properties. For example, the porosity of the bioceramic may vary depending on the silane reactant(s) used. Longer solidification times generally result in the formation of a more porous bioceramic, wherein longer solidification times may be achieved by increasing the amount on non-amino-containing silane reactant(s) relative to the amino-containing silane reactant(s).

These various properties lead to the ability of the bioceramic to be used in a wide range of tissue engineering applications. For example, the bioceramic can be made in scaffolds, which can deliver cells, growth factors, and other additives to a healing site. This can be used to regenerate bone, cartilage, and other tissues. Nano-scaled microstructures can be used to promote cell attachment, growth, and differentiation. Alternatively, the bioceramic may be used to engineer alloplastic grafts. Thus, tissue engineering may be used to replace or augment many natural body tissues. Tissues may be regenerated using these types of structures, and additives may be used to compensate for deficiencies in the patient. Other structures that promote the rapid integration of the bioceramic with the natural tissues may also be used effectively. For example, a structure of the bioceramic may be implanted into a bone, which then acts to stimulate bone regeneration. As another example, the bioceramic may be implanted for cartilage replacement, which may stimulate cartilage regeneration. Still another example relates to the use of the bioceramic for cemented dental implants.

The bioceramic may be produced in different forms, depending upon the intended use and purpose. Suitable forms include solid, putty, paste, and liquid. If the bioceramic is in solid form, it may be, for example, a shaped or unshaped solid, it may be a pre-formed solid, it may be a frame or a lattice, or another solid form. The bioceramic may be formed into a porous scaffold. The solid form may be very stiff, stiff, slightly flexible, soft, rubbery, or other. The bioceramic may be a putty. If in putty form, it may be anywhere from a dense or thin putty. The bioceramic may be a paste. If a paste, it may be anywhere from a thick to a thin paste. If a liquid, it may be from very viscous to very thin.

Due to the wide range of forms in which the bioceramic may be produced, the bioceramic lends itself to a wide range of uses. Uses of the bioceramic include, but are not limited to: for bones, such as for bone graft material, bone cement, or bone replacement; for dental procedures, such as for dental implants, fillings, jaw strengthening or tooth replacement; for joint replacement; for cartilage replacement or reinforcement; for tendon or ligament replacement or repair; and a wide range of tissue engineering applications, including assisting in regenerating bodily tissues.

One application of the bioceramic is to replace bone material in the body. The bioceramic may have properties similar to natural bone. For example, a bioceramic as described herein may have similar strength modulus to natural bone. The benefit of having a similar strength modulus is that biomechanical mismatch problems, such as stress shielding, can be minimized Nanoindentation is a mechanical microprobe method that enables the direct and simultaneous measurement of strength modulus and hardness. The resolution of the test method enables the measurement of bones and materials at a very fine level. Nanoindentation is discussed in more detail in Ko, C. C. et al., Intrinsic mechanical competence of cortical and trabecular bone measured by nanoindentation and microindentation probes, Advances in Bioengineering ASME, BED-29:415-416 (1995). The test may be conducted using an MTS nanoindenter XP (available from MTS Systems Corporation, Eden Prairie, Minn.). The method used may be as described in Chang M. C. et al., Elasticity of alveolar bone near dental implant-bone interfaces after one month's healing, J. Biomech. 36:1209-1214 (2003).

Additionally, the compressive strength of the bioceramic and various natural bones may be tested and compared. A bioceramic may have compressive strength comparable to that of natural bone. A compressive strength test may be conducted using an Instron 4204 Tester (available from Instron Corporation, Canton, Mass.). Tests are conducted according to ASTM C39 “Standard Test Method for Compressive Strength of Cylindrical Concrete Specimens,” and may include using cylindrical samples with height to diameter ratio of 2:1.

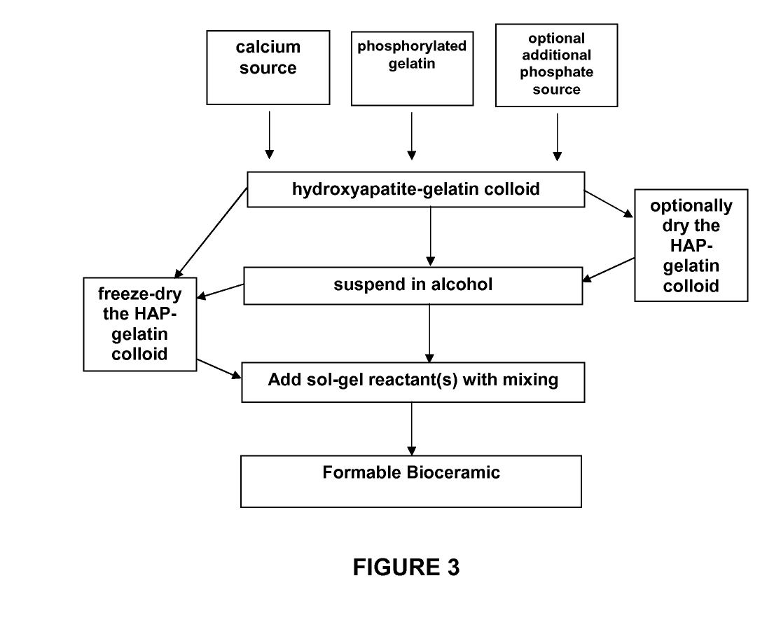

A method for producing a formable bioceramic is described. A flowchart diagram including the major process steps for making a bioceramic described herein is shown in FIGS. 2 and 3. A reactor is setup with temperature control and stirring. A mixture of calcium hydroxide, phosphoric acid, and gelatin is mixed together using a high degree of agitation. These components should be as pure as possible to minimize any contaminants which might weaken the resulting bioceramic. Purchased or produced, the components will preferably be placed into solution prior to use. More preferably, the components will be in an aqueous solution. The various components may be added all at once, or may be added gradually. If added gradually the components in solution may be added using pumps, such as peristaltic pumps (such as Masterflex, available from Cole-Parmer).

The gelatin may be added separately (see FIG. 2), or alternatively, may be pre-mixed together with one of the other components prior to addition. Preferably, the gelatin will be pre-mixed with the phosphoric acid in order to at least partially phosphorylate the gelatin (see FIG. 3). This has been found to lead to better dispersion and growth of the nanocrystals. The gelatin may be dissolved in a solution, and the phosphoric acid added to the solution, or the gelatin may be added to the phosphoric acid and dissolved therein, preferably the latter. In order to assist in dissolving the mixture, the temperature may be controlled between about 35° C. and 40° C., and the mixture stirred during the addition and dissolving. A wide range of gelatin concentrations may be used. Preferably, the concentration will be greater than about 0.001 mmol, greater than about 0.01 mmol, or greater than about 0.025 mmol Preferably, the concentration will be 100 mmol or less, 10 mmol or less, or 1 mmol or less.

In order to enable sufficient phosphorylation of the gelatin, this mixing should continue for some time. Suitably, the mixing will continue for at least about 2 hours. Preferably, the mixture will be mixed for at least about 5 hours. Suitably, the mixing will be continued for less than about 24 hours. Preferably, the mixing will continue for less than about 18 hours, and more preferably less than about 12 hours. It has been found that insufficient mixing time leads to less than a desirable amount of gelatin phosphorylation, and results in larger, less well-dispersed crystals later in the process. When mixed for longer periods, the gelatin begins to lose the ability to react with the other components, with the result that the crystals are no longer held as well by the gelatin later in the process. The ability to hold the crystals and coordinate the gelatin with the hydroxyapatite continues to decline with time, until it decreases sharply after 24 hours of mixing. The obtained intermediate slurries have been found to show different qualities and gelling status based on the phosphorylation time.

After preparation, the calcium, phosphoric acid, and gelatin components (or calcium, phosphorylated gelatin, and optionally additional phosphoric acid) are added together, using agitation and while controlling the pH and temperature. As the components streams are added, co-precipitation begins to occur. This co-precipitation results in the formation of hydroxyapatite nanocrystals in and/or on the gelatin. Preferably, the conditions and component concentrations are maintained such that the continued high-speed agitation and controlled conditions result in the continued formation of hydroxyapatite nanocrystals, rather than the growth of macro-crystals. Under high agitation, this mixture forms a colloidal slurry.

During addition of the components as well as during agitation, the pH of the mixture may be controlled. Suitably, the pH will be controlled to be greater than about 7.0, preferably greater than about 7.5, and more preferably greater than about 7.8. Suitably, the pH will be controlled to be less than about 9.0, preferably less than about 8.5, and more preferably less than about 8.2. The pH may be controlled using the components of the reaction process, using means known in the art. For example, a pH controller (such as Bukert 8280H, available from Bukert) may be used to measure the pH and control the action of the pumps used to add the various components.

The temperature of the mixture may also be controlled during addition of the components and during agitation. Preferably, the temperature will be controlled using a water bath (e.g., as available from Boekel), though many other means of temperature control are also suitable. Suitably, the temperature will be controlled to be greater than about 30° C., preferably greater than about 34° C., more preferably greater than about 36° C. Suitably, the temperature will be controlled to be less than about 48° C., preferably less than about 45° C., and more preferably less than about 40° C. At too low of a temperature, there is insufficient energy to lead to good crystal growth. At too high of a temperature, the crystals grow larger than the desired size.

The co-precipitation is characterized by being a low cost, simple process which is easily applicable and adaptable to industrial production. Moreover, the hydroxyapatite crystals prepared by the co-precipitation generally have the benefits of very small size, low crystallinity, and high surface activation. This enables the bioceramic to meet many different demands.

Properly controlled, the co-precipitation results in a uniform dispersion of hydroxyapatite nanocrystals. Suitably, calcium and phosphate will be present in sufficient amounts to enable the formation and growth of hydroxyapatite nanocrystals. Preferably, the ratio of the number of moles of calcium to the number of moles of phosphate present (as free phosphate and/or phosphorylated gelatin) will be from about 1.5 to about 2.0, more preferably present in a ratio from about 1.6 to about 1.75, and most preferably from about 1.65 to about 1.70. The nanocrystals formed may be needle-shaped, plate-shaped, or may have other crystal shapes. Preferably, hydroxyapatite crystals formed will be needle-shaped.

After addition of all of the components into the co-precipitation reaction, agitation is stopped. The hydroxyapatite-gelatin slurry may be concentrated using centrifugation to remove excessive water. Thereafter, the hydroxyapatite-gelatin colloidal residue may be resuspended in alcohol at a ratio of 0.1 to 100 (alcohol to water removed during concentration), preferably 1:1, followed by centrifugation to yield a hydroxyapatite-gelatin colloidal residue in alcohol. The alcohol may be a straight-chained or branched C₁-C₄ alcohol (e.g., methanol, ethanol, propanol, butanol), a C₂-C₄ diol, and polyvinyl alcohol. Preferably, the alcohol includes methanol. Alternatively, glycerin may be used in place of, or in combination with the alcohol.

The forming process is based on a sol-gel reaction that includes hydrolysis and condensation. Importantly, the method does not require a powder drying process as required by other processes known in the art, however, a dry hydroxyapatite-gelatin colloid may be desirable depending on the desired product and the processing conditions. The hydroxyapatite-gelatin colloidal residue in alcohol is transferred to another reaction flask, setup with high-speed stirring and temperature control. One or more sol-gel, e.g., silane, reactants and optionally at least one inactive filler and/or other additive is added to the flask with vigorous stirring at temperature in a range from about −30° C. to about 30° C. Following cessation of stirring, the mixture is allowed to solidify for a sufficient time, for example, the time of solidification may be in a range from about 1 min to about 1 hr, preferably about 1 min to about 30 min. Preferably, the sol-gel, e.g., silane, reactant(s) include at least one amino-containing silane compound and the gelatin:sol-gel reactant(s) ratio is in a range from about 10 to about 0.1, depending on the desired mechanical strength of the bioceramic product.

The at least one sol-gel reactant may be added in various amounts, depending upon the desired properties of the bioceramic, and the concentration of the other components. The sol-gel reactant(s) may be added directly, or more preferably, will be added as an aqueous solution or mixture. The amount will be selected in order to assist in achieving a bioceramic having the desired properties. The sol-gel reactant(s) may be added to the other components all at once or over a period of time. As introduced hereinabove, preferably the at least one sol-gel reactant includes an amino-containing silane reactant. That said, the inclusion of non-amino-containing silane reactant(s) slows the sol-gel reaction and results in a more porous and more manageable bioceramic.

Following solidification, water may be removed from the sol-gel-based hydroxyapatite-gelatin biomaterial. For example, water may be removed (a) at room temperature and atmospheric pressure, which may take anywhere from about 2 hr to about 12 hr to dry depending on the temperature and humidity, (b) at elevated temperature and atmospheric pressure to drive the water off more quickly, (c) under supercritical conditions using a supercritical fluid, e.g., CO₂, as a drying agent as understood by one skilled in the art; or (d) using an enclosed space with a desiccant under reduced pressure. Abundant ion-exchanged, double-distilled water may be used to wash the biomimetic nanocomposite prior to drying.

A product or shape may be formed from the damp bioceramic (prior to drying), or the bioceramic can be dried without being formed into a shape. The damp material or damp shapes may be stored for later use, or may be dried. The shaped or unshaped bioceramic, damp or dried, may be stored for later use, as the bioceramic is stable in normal atmosphere. Additionally, products may later be cut or shaped from the unformed and unshaped bioceramic.

Optionally, other components or additives, such as described earlier in this application, may be added to the bioceramic. The components may be added during the process, and at any stage, from the initial step to the last step. In addition, the other components may be added to the final bioceramic, whether damp or dry, and whether unformed or formed.

In another aspect, the hydroxyapatite-gelatin material described herein may be freeze dried and subsequently mixed with the at least one sol-gel, e.g., silane, reactant(s) as described herein. A process using the dried hydroxyapatite-gelatin material has the advantage of minimizing bioceramic preparation time when time is of the essence, for example, during surgical procedures. Specifically, the process of this aspect includes making the hydroxyapatite-gelatin slurry as described herein and freeze-drying the slurry to form a hydroxyapatite-gelatin dry powder having a density in a range from about 0.1 g mL⁻¹ to about 0.8 g mL⁻¹. In one alternative, the slurry can be concentrated with or without alcohol prior to freeze-drying. The hydroxyapatite-gelatin powder is preferably pulverized and transferred to another reaction flask, setup with high-speed stirring and temperature control. One or more sol-gel, e.g., silane, reactants and optionally at least one inactive filler and/or other additive is added to the flask with vigorous stirring at temperature in a range from about −30° C. to about 30° C. Following cessation of stirring, at least one aliquot of a buffer solution may be added following which the mixture is allowed to solidify for a sufficient time, for example, the time of solidification may be in a range from about 1 min to about 1 hr, preferably about 1 min to about 30 min. For example, hydroxyapatite-powder may be mixed with the sol-gel reactant for sufficient time, followed by the addition of an aliquot of buffer solution, followed by the addition of a second aliquot of buffer solution. The resulting material is hand moldable and may be used as a biomimetic cement.

Buffer solutions include, but are not limited to, phosphate buffered saline (PBS), lower molecular weight polyalkenoic acid (5-50 wt %), and hyaluronic acid (5-50 wt %). The at least one sol-gel reactant may be added in various amounts, depending upon the desired properties of the moldable bioceramic, and the concentration of the other components. The sol-gel reactant(s) may be added directly, or more preferably, will be added as an aqueous solution or mixture. The amount will be selected in order to assist in achieving a moldable bioceramic having the desired properties. The sol-gel reactant(s) may be added to the other components all at once or over a period of time. As introduced hereinabove, preferably the at least one sol-gel reactant includes an amino-containing silane reactant. That said, the inclusion of non-amino-containing silane reactant(s) slows the sol-gel reaction and results in a more porous and more manageable bioceramic.

A product or shape may be formed from the damp moldable bioceramic (prior to drying), or the bioceramic can be dried without being formed into a shape. The damp material or damp shapes may be stored for later use, or may be dried. The shaped or unshaped bioceramic, damp or dried, may be stored for later use, as the bioceramic is stable in normal atmosphere. Additionally, products may later be cut or shaped from the unformed and unshaped bioceramic.

In yet another aspect, a method of making a sol-gel-based hydroxyapatite-collagen bioceramic using a sol-gel reaction that includes hydrolysis and condensation is contemplated, said method being analogous to the aforementioned method of making a sol-gel-based hydroxyapatite-gelatin bioceramic using the sol-gel reaction.

In another aspect, functional GEMOSOL can be synthesized using the “double encapsulation” technique, wherein trapped agents including, but not limited to, proteins, growth factors, active drugs and living cells are able to be trapped within the GEMOSOL material. The double encapsulation aspect refers to spherical membranes inside the GEMOSOL architecture wherein the membranes include poly(N-isopropylacrylamide, GEMOSOL, or combinations thereof.

The features and advantages are more fully illustrated by the following non-limiting examples, wherein all parts and percentages are by weight, unless otherwise expressly stated.

### Example 1

A moldable bioceramic as described herein was made. Specifically, the hydroxyapatite-gelatin colloidal slurry was prepared and dried and pulverized to form a hydroxyapatite-gelatin powder. Thereafter 0.25 g of the hydroxyapatite-gelatin powder was mixed with 40 μl of enTMOS and mixed for 3 min at room temperature. Then, 200 μL of 1×PBS was added to the mixture for 2 min A second aliquot of 100 μL 1×PBS was added and the resulting material was hand moldable. The quantities mentioned here are subjected to scale up, depending on the need of the applications.

Accordingly, while the invention has been described herein in reference to specific aspects, features and illustrative embodiments of the invention, it will be appreciated that the utility of the invention is not thus limited, but rather extends to and encompasses numerous other aspects, features and embodiments that result from the adsorption-induced tension in molecular (chemical and physical) bonds of adsorbed macromolecules and macromolecular assemblies. Accordingly, the claims hereafter set forth are intended to be correspondingly broadly construed, as including all such aspects, features and embodiments, within their spirit and scope.

1. A formable bioceramic comprising calcium phosphate/gelatin-modified sol-gel (GEMOSOL) nanocomposite.

2. The bioceramic of claim 1, wherein the calcium phosphate comprises hydroxyapatite.

3. (canceled)

4. The bioceramic of claim 1, wherein the GEMOSOL nanocomposite comprises silica.

5. The bioceramic of claim 1, wherein the GEMOSOL nanocomposite comprises phosphorylated gelatin.

6. The bioceramic of claim 1, wherein the calcium phosphate, gelatin and sol-gel components of the bioceramic are substantially dispersed.

7. The bioceramic of claim 4, wherein the calcium phosphate, gelatin and silica components of the bioceramic are substantially dispersed.

8. The bioceramic of claim 1, further comprising at least one additive selected from the group consisting of growth factor, cells, pharmaceutical drugs, anti-inflammatory agents, antibiotics, dyes, and combinations thereof.

9. The bioceramic of claim 8, wherein the growth factor comprises BMP, TGF-β, VEGF, MGP, BSP, OPN, OCN, IGF-I, Biglycan, RANKL, Pro COL-α1, and combinations thereof.

10. The bioceramic of claim 8, wherein the cells comprise osteoblasts, osteoclasts, osteocytes, and/or multipotent stem cells.

11. An article for use in tissue engineering, cemented dental implants or in bone replacement, wherein the article comprises the bioceramic of claim 1.

12. (canceled)

13. (canceled)

14. A method of making a formable bioceramic, said method comprising:
mixing calcium hydroxide, phosphoric acid and gelatin under aqueous conditions to produce a co-precipitated calcium phosphate-gelatin material; and
adding at least one sol-gel reactant to the calcium phosphate-gelatin material to produce a calcium phosphate/gelatin-modified sol-gel (GEMOSOL) nanocomposite.

15. The method of claim 14, wherein the calcium phosphate comprises hydroxyapatite.

16. (canceled)

17. The method of claim 14, wherein the gelatin comprises phosphorylated gelatin.

18. The method of claim 14, wherein the at least one sol-gel reactant comprises at least one silane, wherein the at least one silane reactant comprises a species selected from the group consisting of tetramethylorthosilicate (TMOS), tetraethylorthosilicate (TEOS), 3-aminopropyltrimethoxysilane, bis[3-(trimethoxysilyl)propyl]-ethylenediamine (enTMOS), bis[3-(triethoxysilyl)propyl]-ethylenediamine, methyltrimethoxysilane (MTMS), polydimethylsilane (PDMS), propyltrimethoxysilane (PTMS), methyltriethoxysilane (MTES), ethyltriethoxysilane, dimethyldiethoxysilane, diethyldiethoxysilane, diethyldimethoxysilane, 3-(2-Aminoethylamino)propyltriethoxysilane, N-propyltriethoxysilane, 3-(2-Aminoethylamino)propyltrimethoxysilane, methylcyclohexyldimethoxysilane, dimethyldimethoxysilane, dicyclopentyldimethoxysilane, 3-[2(vinyl benzylamino)ethylamino]propyltrimethoxysilane, 3-aminopropyltriethoxysilane, 3-(aminopropyl)dimethylethoxysilane, bis(3-trimethoxysilylpropyl)-N-methylamine, 3-(aminopropyl)methyldiethoxysilane, 3-(aminopropyl)methyldimethoxysilane, 3-(aminopropyl)dimethylmethoxysilane, N-butyl-3-aminopropyltriethoxysilane, N-butyl-3-aminopropyltrimethoxysilane, N-(β-amimoethyl)-γ-amino-propyltriethoxysilane, 4-amino-butyldimethyl ethoxysilane, N-(2-Aminoethyl)-3-aminopropylmethyldimethoxysilane, N-(2-Aminoethyl)-3-aminopropylmethyldiethoxysilane, 3-aminopropylmethyldiethoxysilane, and combinations thereof.

19. (canceled)

20. The method of claim 14, wherein the at least one sol-gel reactant comprises at least one silane and wherein the at least one silane reactant comprises an amino-containing silane compound.

21. (canceled)

22. (canceled)

23. The method of claim 14, further comprising concentrating the calcium phosphate-gelatin material to remove excess water prior to adding the at least one sol-gel reactant.

24. (canceled)

25. The method of claim 23, further comprising suspending the concentrated calcium phosphate-gelatin material in at least one alcohol prior to adding the at least one sol-gel reactant.

26. The method of claim 25, further comprising concentrating the calcium phosphate-gelatin material to remove excess alcohol or pulverizing the freeze-dried powder prior to adding the at least one sol-gel reactant.

27. (canceled)

28. (canceled)

29. The method of claim 14, further comprising drying the calcium phosphate/gelatin-modified sol-gel (GEMOSOL) nanocomposite.

30. (canceled)

31. A method of making a formable bioceramic, said method comprising mixing calcium phosphate-collagen material with at least one sol-gel reactant to produce a calcium phosphate/collagen-modified sol-gel nanocomposite.

32.-34. (canceled)
